(19) United States
(12) Patent Application Publication
(10) Pub. No.: US 2015/0216683 A1
(43) Pub. Date: Aug. 6, 2015

(54) Prosthetic Distal Force Measurement Device

(71) Applicants: Aldo A. Laghi, Clearwater, FL (US); Eric Prast, St. Petersburg, FL (US); Kevin McLoone, Dunedin, FL (US)

(72) Inventors: Aldo A. Laghi, Clearwater, FL (US); Eric Prast, St. Petersburg, FL (US); Kevin McLoone, Dunedin, FL (US)

(73) Assignee: ALPS Intellectual Property Management, LLC, St. Petersburg, FL (US)

(21) Appl. No.: 14/172,123

(22) Filed: Feb. 4, 2014

(51) Int. Cl. A61F 2/76; A61F 2/78

(52) U.S. Cl. CPC A61F 2/76; A61F 2/7812; A61F 2002/7635

(57) ABSTRACT

An insert interface adapted to fit within a prosthesis between a convex-shaped distal end of a prosthetic liner and an interior distal end of a prosthetic socket for collecting data measurement of the fit of a residual limb of an amputee within the prosthesis comprising an insert comprising a resilient pad of elastomeric material having an upper surface and a lower surface with a concave shaped upper surface adapted to provide a contiguous surface with an interior surface of a prosthetic socket when inserted therein and provide a cushion for a distal end of a residual limb when worn by an amputee, and a lower surface having a shape complementally configured to a distal interior surface of a prosthetic socket when inserted therein, and at least one force sensor mounted centrally within said pad in proximity to the upper surface of the pad for sensing forces against the pad created by a downward movement of a residual limb of an amputee when wearing the socket and for electronically wirelessly transmitting data indicative of said sensed forces via a receiver/transmitter to a remote data processing computer collected by a user, rehabilitation doctor, and/or a CPO (Certified Prosthetist/Orthotist) for determining adjustments to the prosthesis.

### CROSS-REFERENCE TO RELATED APPLICATIONS

Not applicable.

### STATEMENT REGARDING FEDERALLY SPONSORED RESEARCH OF DEVELOPMENT

Not applicable.

### THE NAMES OF THE PARTIES TO A JOINT RESEARCH AGREEMENT

Not applicable.

### INCORPORATION-BY-REFERENCE OF MATERIAL SUBMITTED ON A COMPACT DISC

Not applicable.

### FIELD

This document generally relates to the field of prosthetic liners and sockets having information systems for managing the comfort level of the user.

### BACKGROUND OF THE INVENTION

A lower limb prosthetic liner provides optimum functionality when the distribution of patient's weight in a prosthetic socket is one third distal and two thirds proximal. Currently there is no feasible way for a prosthetist to measure weight distribution during the fitting of a new socket to an amputee. The prosthetist relies on experience to make an approximate evaluation of weight distribution. An inappropriate distribution of weight may result in discomfort, poor gait, and damage to the limb. Many of the lower limb amputees are vascular with poor circulation in the residual limb and poor sensory perception. Excessive pressure points are likely to go unnoticed by the amputees which result in wounds that are difficult to heal. Even if the initial socket is properly made and allows an optimal distribution of weight therein, with the maturing of the residual limb over time, the size of the limb shrinks and the pressure of the distal end of the residual limb against the distal end surface of the socket increases. Vascular patients are subjected to positive and negative volume fluctuations more than healthy individuals and such fluctuations affect the distribution of weight in the socket. This problem is addressed by the prosthetist by training the patient to use additional prosthetic socks or a different ply sock for the management of the volume fluctuations of the residual limb. This solution is only partially effective with many older amputees who are not able to assess how many prosthetic socks or what ply of sock to use.

As a solution to this problem, prosthetic sockets and/or liners now on the market have been designed to have residual limb monitoring systems built into them. For example, Published Patent Application No. US 2012/0226197 to Sanders, et al. disclose a prosthetic liner having sensors built into the liner for monitoring various activities of the amputee that effect the volume of an amputee's residual limb. The sensors on the liner further includes a transmitters for transmitting the collected data to a remote computer system of the user, a doctor or prosthetist for developing a sock monitoring strategy. Such data is useful in determining what adjustments must be made to the interface between the residual limb and socket, such as adding or eliminating extra socks, or using sock of a different ply thickness.

However, to date, such prior art systems either have the sensors built into the liner or into the socket. Such liners and sockets are complicated to manufacture and expensive not to mention the fact that costs rise dramatically if they have to be replaced. Thus, the present invention provides another and simpler solution to this problem without having to mount the sensors in the liner and/or socket. The present invention provides a simple resilient insert having force sensing and transmitting electronics for continuous and accurate measurement of the distal contact of an amputee's residual limb against the inside distal surface of a prosthetic socket.

### BRIEF SUMMARY OF THE INVENTION

The present invention relates generally to a pressure measurement device that is inserted into a prosthetic socket at the distal end and located between the socket and a socket liner. The device comprises a molded replaceable cushion insert made of resilient silicone or polymeric material shaped to have a lower surface to complementally fit firmly against the interior distal end surface of the socket and an upper concave surface that is contiguous with the concave surface of the distal interior of the socket. The insert is removable from the socket and therefore replaceable. The insert further includes at least one force sensor and electronic receiving and transmitting circuitry associated therewith either embedded or removably mounted therein. In the embodiment where the force sensor and circuitry are embedded therein such as during the molding process, the entire device would be replaceable. In the embodiment where the force sensor and circuitry are removably mounted therein, only the force sensor and/or the transmitting circuitry would have to be replaced. Each force sensor is disposed in the insert and adapted to detect downward pressure caused by a distal end of an amputee's residual limb against the distal end of the socket. Throughout the day, an amputee's residual limb may change in volume such as by swelling or contraction of the residual limb for various reasons. Such volumetric changes will affect the fit of the residual limb within the socket which in turn affects the downward pressure of the residual limb against the distal end of the socket. The present invention is designed to measure these pressure variations and provide feedback to the user, rehabilitation doctor, and/or a CPO (Certified Prosthetist/Orthotist). The electronic circuitry transmits the pressure measurements wirelessly to a personal computer or mobile computing device throughout the day. This invention is designed to systematically inform the user, rehabilitation doctor, and/or a CPO (Certified Prosthetist/Orthotist) of the degree of force or pressure that the distal end of the residual limb is exerting against the distal end of the prosthetic socket throughout the time of use. Accordingly, such information is useful in determining whether a new socket, a new sock, additional socks or elimination of socks may be needed.

In a first embodiment, the insert is molded of a resilient silicone or polymeric material to have a concave-convex shape with the convex side molded to match the interior concave surface of a distal end of a socket. This embodiment could be molded to have at least one force sensor and transmitter circuitry embedded therein or molded to have a cavity in the convex side in which the at least one force sensor and transmitting circuitry can be removably secured.

In a second embodiment, the insert is also molded to have a concave-convex shape with the convex side molded to match the interior concave surface of a distal end of a socket. However, in this embodiment, the convex side of the insert includes an extension leading therefrom which is complementally shaped to fit inside a well or recess defined by a lower section of the socket depending from the distal end thereof. This embodiment could be molded to have at least one force sensor and transmitter circuitry embedded in the extension or molded to have a cavity in the extension in which the at least one force sensor and transmitting circuitry can be removably secured.

The above two embodiments are designed to function with a suction liner or a suspension liner, but could also be modified to function with a locking liner. When modified to function with a locking liner, each of the above define inserts could be molded to have a central opening that aligns with an opening in the distal end of the socket for receiving a locking pin therethrough. Such locking pins are conventionally mounted to the distal end of the liner and cooperate with a locking mechanism mounted to the distal end of the socket. In these modified embodiments, the electronic transmitting circuitry could also be modified to have an opening for the locking pin to pass through.

### BRIEF DESCRIPTIONS OF THE DRAWINGS

FIG. 1 shows a cross-sectional view of the first embodiment of the present invention inserted between a prosthetic liner and the distal end of a prosthetic socket with the electronic components embedded within the insert.

FIG. 2 shows a cross-sectional view of the first embodiment of the present invention inserted between a prosthetic liner and the distal end of a prosthetic socket modified to have a central opening for receiving a locking pin of a locking liner.

FIG. 3 is a cross-sectional view of a second embodiment of the present invention inserted between a prosthetic liner and the distal end of a prosthetic socket and having a housing for the receiver/transmitter circuitry separate from the force sensor.

FIG. 4 is a cross-sectional view of the second embodiment of the present invention inserted between a prosthetic liner and the distal end of a prosthetic socket modified to have a central opening for receiving a locking pin of a locking liner.

FIG. 5 is a cross-sectional view of a third embodiment of the present invention inserted between a prosthetic liner and the distal end of a prosthetic socket and having a housing for the receiver/transmitter circuitry separate from the force sensor.

FIG. 5a is an enlarged cross-sectional view of the encircled structure illustrated in FIG. 5 illustrating a first modification of the force sensor and housing for the receiver/transmitter circuitry separate from the force sensor.

FIG. 5b is an enlarged cross-sectional view of the encircled structure illustrated in FIG. 5 illustrating a second modification of the force sensor housing for the receiver/transmitter circuitry separate from the force sensor.

FIG. 5c is an enlarged cross-sectional view of the encircled structure illustrated in FIG. 5 illustrating a third modification of the force sensor and housing for the receiver/transmitter circuitry separate from the force sensor.

FIG. 6 is a cross-sectional view of the third embodiment of the present invention inserted between a prosthetic liner and the distal end of a prosthetic socket modified to have a central opening for receiving a locking pin of a locking liner.

FIG. 7 is a cross-sectional view of the third embodiment shown in FIG. 3 along the lines VII-VII.

FIG. 8 is an illustrated view of how the present invention communicates with computer systems via Bluetooth technology.

### DETAILED DESCRIPTION OF THE INVENTION

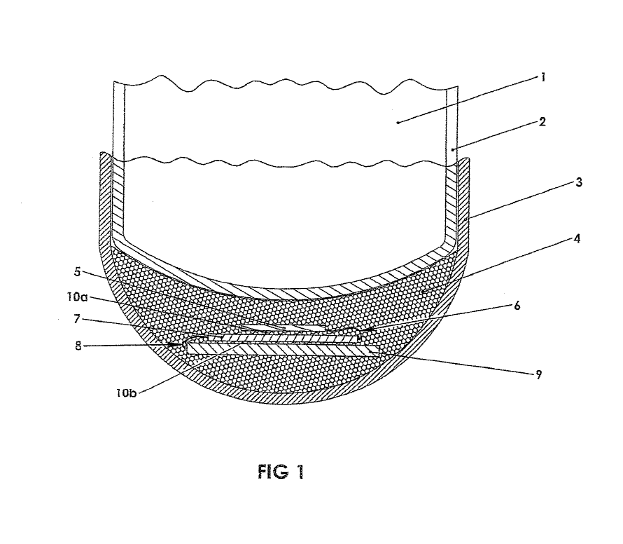

Referring to FIG. 1, a residual limb of an amputee is denoted by the numeral 1. As illustrated, prosthetic liner 2 is donned onto the residual limb and inserted into prosthetic socket 3. A first embodiment of the present invention includes an insert 4 comprising elastomeric member adapted to fit between the distal end of the liner and distal interior of the socket. Embedded within the elastomeric member 4 is force sensor 5 electrically connected to data collecting and transmitting circuitry 7 via conductors 6, and battery 9 electrically connected to circuitry 7 via conductors 8. The insert 4 is removable and therefore replaceable should failure of the force sensor or any electronic component be necessary, or simply because the elastomeric member itself has reached its fatigue state.

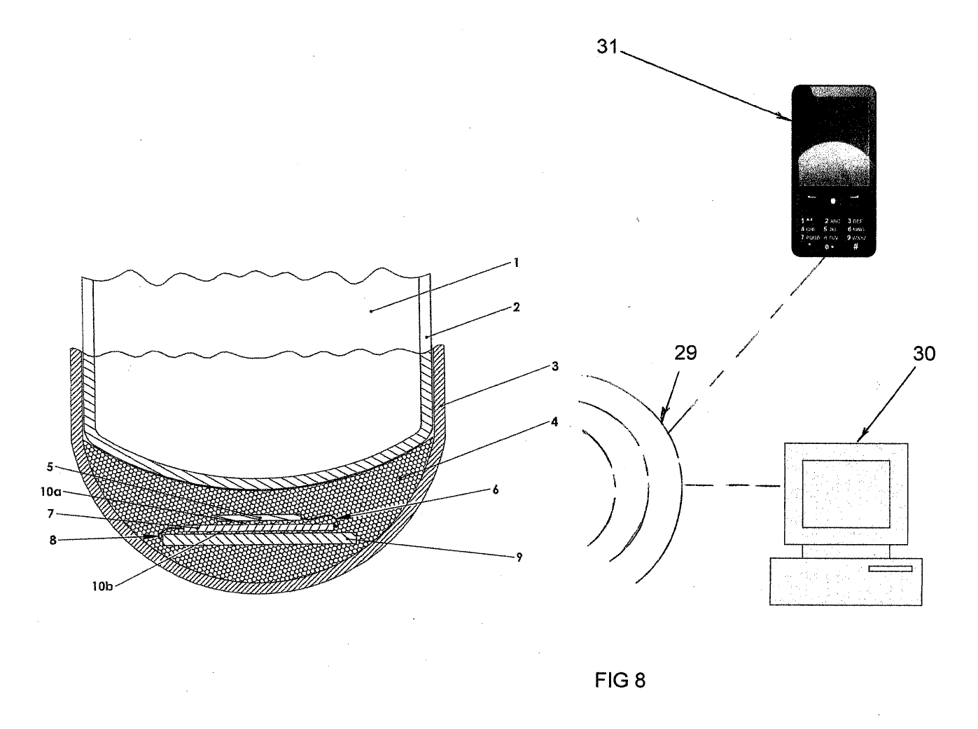

The present invention insert 4 is not limited for use with any particular prosthetic liner and socket arrangement. The embodiment of FIG. 1 could be employed with any conventional suction and or suspension type of prostheses as, for example, those disclosed in U.S. Pat. No. 6,544,292; 5,314,497; 5,571,208; 4,479,272 or 8,523,951, all incorporated herein by reference. Although not shown, the socket of the embodiment of FIG. 1 could be connected to a pylon arrangement for a prosthetic foot as, for example, illustrated in FIG. 7 of U.S. Pat. No. 5,888,217, incorporated herein by reference.

The insert 4 is made of a thermoplastic elastomeric material, preferably gel materials, for example by injection molding techniques, such as silicone, copolymer Styrenic gels, polyurethane, block copolymers or other TPE elastomers. A wide variety of thermoplastic materials that could be used to manufacture the present invention are disclosed in U.S. Pat. No. 5,633,286, incorporated herein by reference.

The force sensor 5 is comprised of an ultra-thin flexible force sensing element used to measure a relative change in force or applied load. It may be used for measuring rate of change and identify force thresholds to trigger an appropriate action. The sensor may be also used as a means of switching a device on therefore detecting presence contact and/or touch. The device used in this design is a durable piezoresistive force sensor created in various shapes and sizes tailored to the gel apparatus. The resistance measured is inversely proportional to the applied force. One type of force sensor is the FlexiForce® sensor manufactured and patented by Tekscan, this type of sensor provides a linear force measurement ±3% and can withstand high temperature environments up to 400° F. (HT201). The FlexiForce® sensor can measure up to 100 lbs of force with <5 microseconds response time. The sensor 5 is connected through pins to a flexible conductive fabric, thread or elastic bonded wire 6 which carries the signal to a receiver/transmitter microcontroller 7. The force sensor cables may also contain a resistor or resistive device to provide a ground reference to the controller.

The receiver/transmitter microcontroller 7 receives data from the force sensor 5 and relays such data via Bluetooth technology 29 to an external electronic device such as a personal computer 30 or cell phone 31 as illustrated in FIG. 8. The receiver/transmitter microcontroller 7 may include an RN41 which is a small form factor, low power; class 1 Bluetooth module that operates on the 2.4 GHz ISM band with a maximum distance of 100 M. The RN41 delivers up to 3 Mbps data rate and uses FHSS/GFSK modulation with 79 channels at 1 MHz intervals. The module uses a 128 bit encryption key for secure communications and uses a UART local over the air RF-configuration. This device has auto-discovery/pairing without software configuration. It provides an auto-connector master, IO pin (DTR line) and character based trigger modes. Although one type of receiver/transmitter microcontroller is described, the present invention is not so limited and will operate with any other conventional receiver/transmitter microcontroller.

One of the main advantages of this RF module and other types are the ultra-low power sleep mode that provides efficient battery use while asleep. The module will maintain a heartbeat looking for a control signal to wake up and transmit data again. At the receiving data may be processed, saved, cataloged, and displayed to the end user. The EEDS may be a hand-held electronic device or software application, the software is designed to be compatible with Android, IOS, or other major smart-phone device operating systems. The software application is an integral component of the system. The software is necessary in order to record a historical trend of the patients fit as well as perform sensor calibration. The EEDS can be also used by the clinician to provide feedback on the socket fit. The software also allows the ability to set how many data-points per day, and will generate a report and send it through email. The EEDS also has the ability to alert the patient of low battery levels in the device.

The battery 9 is a power supply of the PSBL type and may be a lithium ion, lithium polymer, lithium iron phosphate, nickel-cadmium or any rechargeable energy source. A single supply is used with an on-board voltage regulator to power 1.8V, 3.3V, and 5V levels. The cells may be configured in a single or multiple parallel, series, or similar layout.

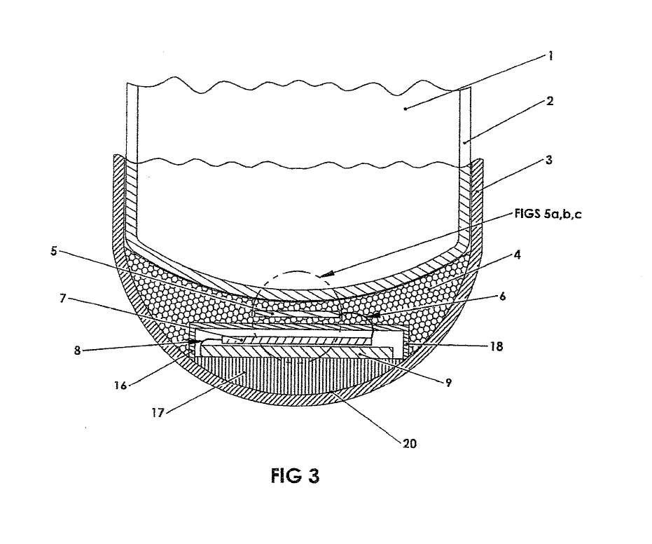

As a wearable device battery life, energy density, accessibility and rechargeable capabilities are essential. In the first embodiment, the force sensor, electronic circuitry and battery are adhered to each by adhesive layers 10a and 10b and fully embedded within the elastomeric material 4 during the molding process. Thus, the entire unit may be disposable or, if not, may include a conventional recharging circuit (not shown) by plugging in the apparatus through a charging port. In the second and third embodiments illustrated in FIGS. 3-6, the battery is removable and can either be replaced or recharged using any conventional recharging circuit.

All of the embodiments disclosed herein could be molded in various sizes to fit different size sockets or could be custom fitted to the distal interior surface of a prosthetic socket that may be in use by the user.

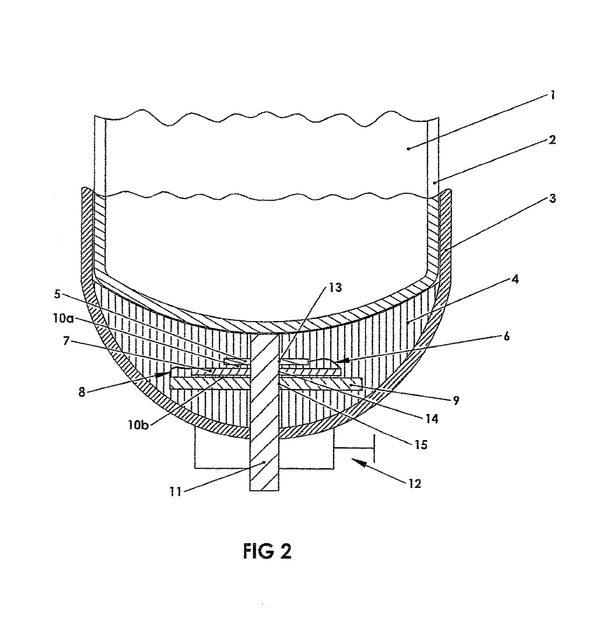

Referring to FIG. 2, a modification to the embodiment shown in FIG. 1 is illustrated. Liner 2 may have a locking pin 11 attached to the distal end thereof. Each of the sensor 5, circuitry 7 and battery 9 include a central opening 13, 14 and 15, respectively, for permitting a locking pin 11 to pass therethrough and be locked in place by a locking mechanism 12. Although the specifics of the locking arrangement is not shown, any conventional locking liner arrangement, such as that disclosed in U.S. Pat. No. 8,394,150, could be modified to include the present invention.

Referring to FIG. 3, a second embodiment of the present invention is illustrated. This embodiment is similar to the embodiment of FIG. 1 with the exception of including a housing 16 for the circuitry 7 and battery 9. The housing 16 could be molded together with the insert 4, or insert 4 could be molded to have a cavity 18 for receiving the housing 16. Whether the housing is molded with the insert, or fitted into the cavity, the bottom edge of the housing sidewall are disposed to rest against the interior surface of the distal end of the socket. This embodiment further includes cap 17 adapted to fit into the lower end of the housing and has a lower surface 20 complementally configured to the distal interior surface of the socket to provide support for the circuitry and battery in the housing. Thus, in this embodiment, the circuitry 7 and battery 9 can be removed from the housing for servicing, or the housing with the circuitry and battery therein can be removed from the insert 4 for servicing.

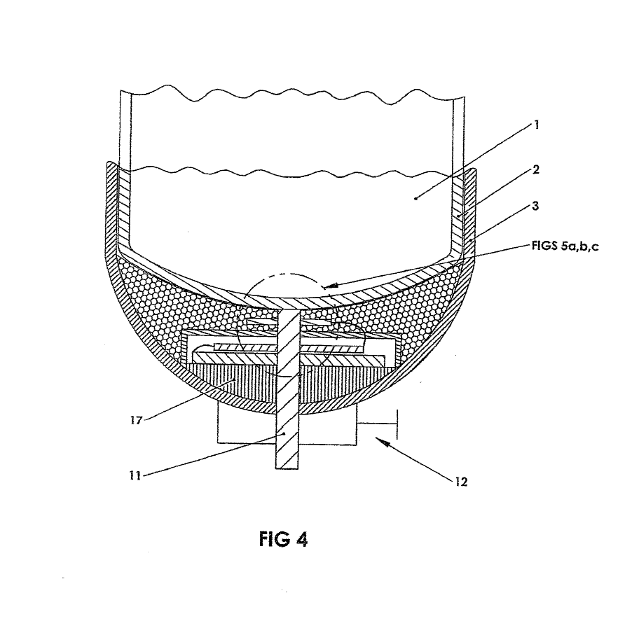

Referring to FIG. 4, a modification to the embodiment shown in FIG. 3 is illustrated. As in the embodiment of FIG. 2, liner 2 may have a locking pin 11 attached to the distal end thereof. Each of the sensor 5, circuitry 7 and battery 9 include a central opening 13, 14 and 15, respectively, for permitting a locking pin 11 to pass therethrough and be locked in place by a locking mechanism 12.

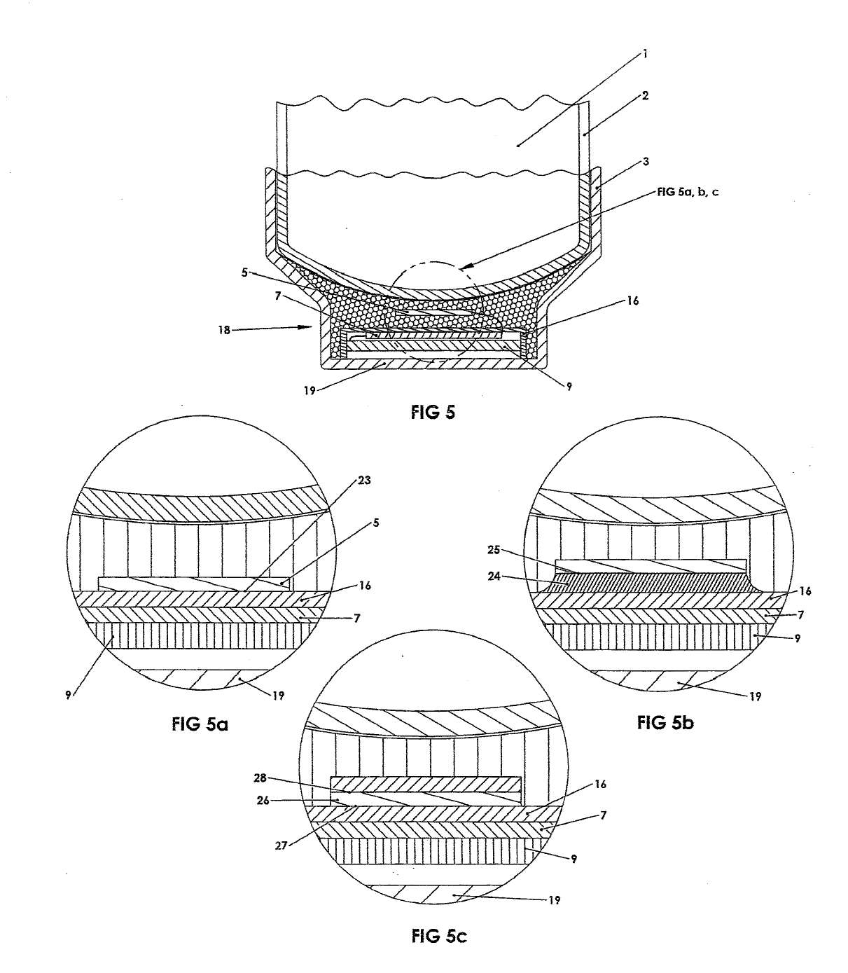

Referring to FIG. 5, a third embodiment of the present invention is illustrated. This embodiment is similar to the embodiment of FIG. 3 with the exception of including a well or recess 18 extending from the lower end of the socket. The well or recess 18 could have any cross-sectional shape, such as square or circular. The well or recess terminates in a flat bottom surface 19 on which the lower edge of housing 16 is supported. Therefore, this embodiment does not require a cap as in the embodiment of FIG. 3. All the other features of this embodiment are the same as in the embodiment of FIG. 3 and thus, will not be repeated here.

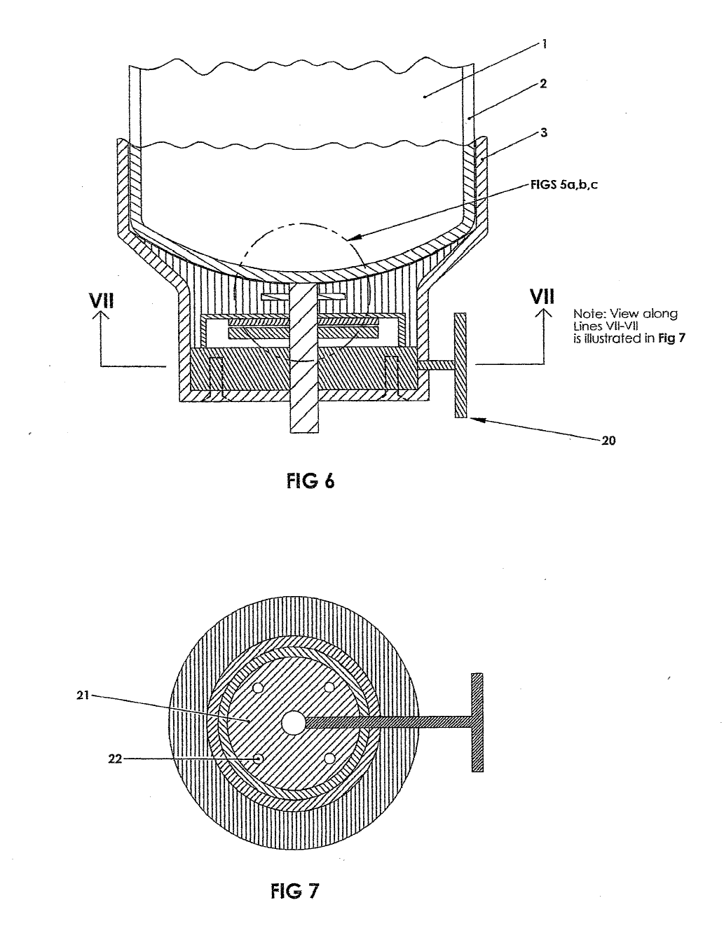

Referring to FIG. 6, a modification to the embodiment shown in FIG. 5 is illustrated. As in the embodiments of FIGS. 2 and 4, liner 2 may have a locking pin 11 attached to the distal end thereof. Each of the sensor 5, circuitry 7 and battery 9 include a central opening 13, 14 and 15, respectively, for permitting a locking pin 11 to pass therethrough and be locked in place by any conventional locking mechanism 20 such as that disclosed in U.S. Pat. No. 8,444,702. It is noted that in this embodiment, the locking mechanism 20 is mounted within the socket whereas the locking mechanism of the embodiments of FIGS. 2 and 4 are mounted exterior to the socket.

In FIG. 7, a cross-sectional view of the cross-section through lines VII-VII in FIG. 6 is illustrated. The locking mechanism may include a plate 21 having screw holes 22 for fasteners such as screws (not shown) for securing the locking mechanism and also may be used to attach a pedestal (not shown) for attaching a pylon and an artificial foot prosthesis (not shown).

Referring to FIGS. 5 a, b and c, three possible modifications of the embodiments of FIGS. 3, 4, 5 and 6 are illustrated. The force sensor 5 in the embodiments of FIGS. 3, 4, 5 and 6 is shown detached from the housing 16 and is embedded within the insert 4. The force sensor 5 is electrically connected to circuitry 7 via conductor 6. Thus, the conductor 6 must extend within the housing sufficiently in order to remove the circuitry 7 and battery 9 from the housing 16 for servicing. On the other hand, should the housing with circuitry and battery therein be removed from the cavity 18 in the insert 4, the conductor 6 must be disconnected from the circuitry in order to service the circuitry and battery. In all three modifications shown in FIGS. 5 a, b and c, the force sensor 5 is attached to the housing 16. In FIG. 5a, the force sensor is attached directly to the top surface of housing 16 by adhesive layer 23. In FIG. 5b, the top surface of the housing is formed with a boss 24 to which sensor 5 is attached by an adhesive layer 25. In FIG. 5c, a separate plastic sheet 26 is mounted to the top surface of the housing by adhesive layer 27 to which the sensor 5 is attached by adhesive layer 28. The boss 24 in FIG. 5b and plastic piece 26 in FIG. 5c provide extra backing for the sensor 5 and also dispose the sensor 5 more approximate to the bottom of the liner 2 rendering it more sensitive to downward pressure from the residual limb.

The invention has been described in terms of various embodiments. It will be appreciated by those skilled in the art that various changes and modifications may be made to the embodiments without departing from the spirit or scope of the invention. It is not intended that the invention be limited to the embodiments shown and described. It is intended that the invention include all foreseeable modifications to the embodiments shown and described. It is intended that the invention be limited in scope only by the claims appended hereto.

What is claimed is:

1. An insert interface adapted to fit within a prosthesis between a convex-shaped distal end of a prosthetic liner and an interior distal end of a prosthetic socket for collecting data measurement of the fit of a residual limb of an amputee within the prosthesis comprising:
said insert comprising a resilient pad of elastomeric material having an upper surface and a lower surface;
said upper surface having a concave shape adapted to provide a contiguous surface with an interior surface of a prosthetic socket when inserted therein and provide a cushion for a distal end of a residual limb when worn by an amputee;
said lower surface having a shape complementally configured to a distal interior surface of a prosthetic socket when inserted therein;
said insert being sized and configured to be disposed within the a prosthetic socket only between a distal end thereof and the convex-shaped distal end of a prosthetic liner;
at least one force sensor mounted centrally within said pad in proximity to said upper surface for sensing forces against said pad created by a downward movement of a residual limb of an amputee when wearing said socket and for electronically transmitting data indicative of said sensed forces;
a receiver mounted within said pad electrically communicating with said at least one force sensor for electronically receiving said data from said at least one force sensor;
a transmitter mounted within said pad electrically communicating with said receiver for wirelessly electronically transmitting said data from said receiver to a remote data processing computer;
a power source mounted within said pad electrically connected to said at least one force sensor, receiver and transmitter for powering the same;
whereby said data is collected by a user, rehabilitation doctor, and/or a CPO (Certified Prosthetist/Orthotist) for determining adjustments to the prosthesis.

2. An insert interface as claimed in claim 1, wherein said elastomeric material comprises a silicone, polyurethane, copolymer or block copolymer.

3. An insert interface as claimed in claim 1, wherein said at least one force sensor, receiver, transmitter and power source are embedded within said elastomeric material.

4. An insert interface as claimed in claim 3, wherein said at least one force sensor, receiver, transmitter and power source are secured to each other by an adhesive.

5. An insert interface as claimed in claim 3, wherein said at least one force sensor is separated from said receiver, transmitter and power source.

6. An insert interface as claimed in claim 5, wherein said receiver, transmitter and power source are secured to each other by an adhesive.

7. An insert interface as claimed in claim 1, wherein said at least one sensor is embedded within said elastomeric material and said receiver, transmitter and power source are removably mounted within said elastomeric material.

8. An insert interface as claimed in claim 7, wherein said insert has a cavity formed in its lower surface; a rigid housing removably secured in said cavity; wherein said receiver, transmitter and power source are removably mounted in a housing.

9. An insert interface as claimed in claim 8, wherein said housing includes a top surface, side walls and a lower cap for enclosing said receiver, transmitter and power source, said housing side walls having a lower edge intersecting the lower surface of said pad whereby said housing and pad are supported by an interior distal surface of a socket when inserted therein.

10. An insert interface as claimed in claim 8, wherein said housing includes a top surface, side walls and an open bottom for enclosing said receiver, transmitter and power source, said housing side walls having a lower edge intersecting the lower surface of said pad whereby said housing and pad are supported by an interior distal surface of a socket when inserted therein.

11. An insert interface as claimed in claim 1, wherein said at least one sensor, receiver, transmitter and power source are removably mounted within said elastomeric material.

12. An insert interface as claimed in claim 11, wherein said insert has a cavity formed in its lower surface; a rigid housing removably secured in said cavity; wherein said receiver, transmitter and power source are removably mounted in a housing.

13. An insert interface as claimed in claim 12, wherein said housing includes a top surface, side walls and a lower cap for enclosing said receiver, transmitter and power source, said housing side walls having a lower edge intersecting the lower surface of said pad whereby said housing and pad are supported by an interior distal surface of a socket when inserted therein.

14. An insert interface as claimed in claim 12, wherein said housing includes a top surface, side walls and an open bottom for enclosing said receiver, transmitter and power source, said housing side walls having a lower edge intersecting the lower surface of said pad whereby said housing and pad are supported by an interior distal surface of a socket when inserted therein.

15. An insert interface as claimed in claim 13, wherein said at least one sensor is adhered directly to the top surface of said housing.

16. An insert interface as claimed in claim 14, wherein said at least one sensor is adhered directly to the top surface of said housing.

17. An insert interface as claimed in claim 13, wherein said top surface of said housing includes a boss member and said at least one sensor is adhered directly to said boss member, whereby said boss member provides added backing support for said force sensor.

18. An insert interface as claimed in claim 14, wherein said top surface of said housing includes a boss member and said at least one sensor is adhered directly to said boss member, whereby said boss member provides added backing support for said force sensor.

19. An insert interface as claimed in claim 13, wherein a rigid plastic sheet is adhered to said top surface of said housing and said at least one sensor is adhered to said rigid plastic sheet, whereby said rigid plastic sheet provides added backing support for said force sensor.

20. An insert interface as claimed in claim 14, wherein a rigid plastic sheet is adhered to said top surface of said housing and said at least one sensor is adhered to said rigid plastic sheet, whereby said rigid plastic sheet provides added backing support for said force sensor.

21. An insert interface as claimed in claim 3, wherein said insert includes a central passage for the passage of a locking pin of a locking liner and said at least one sensor, receiver, transmitter and power source are disposed in said pad around said central passage.

22. An insert interface as claimed in claim 9, wherein said insert includes a central passage for the passage of a locking pin of a locking liner and said at least one sensor, said housing, said receiver, said transmitter and said power source in said housing are disposed around said central passage.

23. An insert interface as claimed in claim 10, wherein said insert includes a central passage for the passage of a locking pin of a locking liner and said at least one sensor, said housing, said receiver, said transmitter and said power source in said housing are disposed around said central passage.

24. An insert interface as claimed in claim 15, wherein said insert includes a central passage for the passage of a locking pin of a locking liner and said at least one sensor, said housing, said receiver, said transmitter and said power source in said housing are disposed around said central passage.

25. An insert interface as claimed in claim 19, wherein said insert includes a central passage for the passage of a locking pin of a locking liner and said at least one sensor, said rigid plastic sheet, said housing, said receiver, transmitter and power source in said housing are disposed around said central passage.

26. An insert interface as claimed in claim 17, wherein said insert includes a central passage for the passage of a locking pin of a locking liner and said at least one sensor, said boss member, said housing, said receiver, transmitter and power source in said housing are disposed around said central passage.

27. An insert interface as claimed in claim 16, wherein said insert includes a central passage for the passage of a locking pin of a locking liner and said at least one sensor, said housing, said receiver, said transmitter and said power source in said housing are disposed around said central passage.

28. An insert interface as claimed in claim 20, wherein said insert includes a central passage for the passage of a locking pin of a locking liner and said at least one sensor, said rigid plastic sheet, said housing, said receiver, transmitter and power source in said housing are disposed around said central passage.

29. An insert interface as claimed in claim 18, wherein said insert includes a central passage for the passage of a locking pin of a locking liner and said at least one sensor, said boss member, said housing, said receiver, transmitter and power source in said housing are disposed around said central passage.
